(19) United States
(12) Patent Application Publication
(10) Pub. No.: US 2014/0147937 A1
(43) Pub. Date: May 29, 2014

(54) METHOD OF DETERMINING ACTIVE CONCENTRATION

(71) Applicants: Robert Karlsson, Uppsala (SE); Hakan Roos, Uppsala (SE)

(72) Inventors: Robert Karlsson, Uppsala (SE); Hakan Roos, Uppsala (SE)

(21) Appl. No.: 14/130,027

(22) PCT Filed: Jun. 27, 2012
(86) PCT No.: PCT/SE2012/050718

§ 371 Date: Dec. 30, 2013

(30) Foreign Application Priority Data
Jun. 30, 2011 (SE) 1150613.6

(51) Int. Cl. G01N 33/543

(52) U.S. Cl. CPC G01N 33/54306
USPC 436/501

(57) ABSTRACT

A method of determining active concentration of an analyte in a sample comprises the steps of:

(a) contacting a laminar flow of the sample with a plurality of solid phase surfaces or surface area supporting a ligand capable of specifically binding the analyte, each surface or surface area having a different ligand density,
(b) determining the initial binding rate (dR/dt) of analyte to the ligand at each ligand-supporting surface or surface area,
(c) from the determined initial binding rates determining the initial binding rate corresponding to transport-limited interaction at the surfaces or surface areas, and
(d) from the initial binding rate determined in step (c) determining the active analyte concentration.

### FIELD OF THE INVENTION

The present invention relates to the determination of the concentration of a bioanalyte, such as a protein, and more particularly to the determination of the active concentration of the bioanalyte.

### BACKGROUND OF THE INVENTION

There are numerous ways to determine the concentration of proteins and other biomolecules, the majority of the methods involving comparison of the sample to a standard preparation. In many cases, however, no standard is available or the activity of the standard is uncertain.

Many times it is also of importance to know the active concentration of bioanalytes rather than the total concentration which may include functionally inactive molecules. This is, for instance, the case in the development and production of biotherapeutics. However, many established methods for measurement of protein concentration do not distinguish between active and inactive molecules.

Thus, whereas the total concentration of e.g. a protein is typically measured by UV or NIR absorption spectrometry which do not distinguish between active and inactive molecules, the active concentration of a biomolecule may conveniently be measured by biosensor technology, wherein a sample containing the biomolecule is contacted with a sensor surface with a specific ligand immobilized thereon, and the association/dissociation process at the surface is monitored. In this case it is the choice of ligand that defines the activity being measured.

Conventionally, active concentration is measured using a calibration curve. In a development of the determination of active concentration using biosensor technology, however, the analyte concentration can be determined without reference to a calibration standard, using the relationship between the diffusion properties of the analyte and the analyte concentration. Thus, if the diffusion coefficient of the analyte is known, the analyte concentration can be calculated. This type of concentration measurement, which can be useful when no satisfactory calibrant is available for an analyte under study, is usually referred to as Calibration-Free Concentration Analysis (CFCA), and relies upon measurement of analyte binding at varying flow rates under conditions where the observed rate of binding is partially or completely limited by transport of analyte molecules to the sensor surface, i.e. partially or completely controlled by diffusion.

Using surface plasmon resonance detection, CFCA was first described by Karlsson, R., et al. (1993), J. Immunol. Methods 166(1):75-8, and further by Sigmundsson, K., et al. (2002) Biochemistry 41(26):8263-76. This methodology has been implemented in the commercial Biacore® systems (marketed by GE Healthcare, Uppsala, Sweden).

In the Biacore® instruments, samples are injected on a micro-flow system and transported by laminar flow to the sensor surface. Molecules reach the sensor surface from bulk solution by a diffusion controlled transport process. In addition to the concentration of analyte molecules, factors influencing the transport rate include the diffusion coefficient, flow cell dimensions, and flow rate. The balance between the transport rate and the binding rate determines whether the observed binding will be transport limited or reaction limited. For successful CFCA, the observed binding rate must, as mentioned above, be at least partially limited by transport.

The current implementation of the technology in Biacore® systems involves two binding experiments where analyte binds to immobilized ligand; one experiment at a low flow rate (often 5 or 10 μl/min) and one experiment at a high flow rate (often 100 μl/min). In the analysis, responses are checked for transport (diffusion) limited behavior and the response is fitted to a kinetic model where the transport coefficient is constant and the concentration of the analyte is fitted. (For details it is referred to 28-9768-788A Biacore T200 Software Handbook (GE Healthcare, Uppsala, Sweden)).

This approach although efficient in many cases has several limitations. Firstly, the degree of transport limitation is highly dependent on the immobilization level where too low immobilization levels result in data with too little transport limitation, and too high immobilization levels may lead to hook effects (i.e. the response is reduced above a certain immobilization level). Secondly, the dynamic range, i.e. the concentration range over which the assay is useful, is limited. Further, the method is time-consuming since two cycles/sample concentration are required. Also, data analysis is based solely on kinetic algorithms.

It is an object of the present invention to provide a novel approach to improve the reliability of CFCA measurements under partial or complete transport limitation.

### SUMMARY OF THE INVENTION

The above mentioned object as well as other objects and advantages are obtained by a method for determination of active concentration using (at least) a single flow pass, or injection, over several sensor surfaces or surface areas with varying ligand density (i.e. an array of different ligand densities) and a curve fitting algorithm for determining the maximum initial binding rate and thereby the active concentration. The procedure is direct and requires no standard curve.

In one aspect, the present invention therefore provides a method of determining active concentration of an analyte in a sample, comprising the steps of:

(a) contacting a laminar flow of the sample with a plurality of solid phase surfaces or surface areas supporting a ligand capable of specifically binding the analyte, each surface or surface area having a different ligand density,
(b) determining the initial binding rate (dR/dt) of analyte to the ligand at each ligand-supporting surface or surface area,
(c) from the determined initial binding rates in step (b) determining the initial binding rate corresponding to transport-limited interaction at the solid phase surfaces or surface areas, and
(d) from the initial binding rate determined in step (c) determining the active analyte concentration.

The initial binding rate determined in step (c) is typically the maximum binding rate.

In a preferred embodiment, however, the method comprises determining the initial binding rates for two different flow rates, determining from initial binding rate ratios for the two flow rates at the different ligand densities, the lowest ligand density where the initial binding rate is proportional to the cubic root of the flow rate, and from this initial binding rate determining the active analyte concentration.

In a variant of the above preferred embodiment, the flow rate is varied during a single contacting (e.g. injection) cycle.

In another preferred embodiment, the method further comprises co-evaluating data from determination of active concentration by kinetic analysis at different flow rates under partial or complete transport limitation, where binding data are fitted to a kinetic model where the transport coefficient is constant. Preferably, several dilutions of the liquid sample are used and included in a global fit of the binding data.

Other preferred embodiments are set forth in the dependent claims.

The method of the invention may conveniently be implemented by software run on an electrical data processing device, such as a computer. Such software may be provided to the computer on any suitable computer-readable medium, including a record medium, a read-only memory, or an electrical or optical signal which may be conveyed via electrical or optical cable or by radio or other means.

Another aspect of the invention therefore relates to a computer program product comprising instructions for causing a computer to perform the method steps of any one of the above-mentioned method variants.

A more complete understanding of the present invention, as well as further features and advantages thereof, will be obtained by reference to the following detailed description and the accompanying drawings.

### BRIEF DESCRIPTION OF THE DRAWINGS

FIGS. 1A to D are different graphs obtained in simulation of initial binding rates.

FIG. 2 is a plot of fitted binding data at eight different binding levels.

FIG. 3 is a plot of initial binding rate versus immobilization level.

FIG. 4 is a plot of binding versus time at two immobilization levels where the flow rate was changed during the injection.

FIG. 5 is corresponding plot to that of FIG. 4 prepared for analysis.

FIG. 6 is a plot of fitted binding versus time data for two different flow rates in conventional CFCA.

FIG. 7 is a plot of globally fitted binding versus time data at several concentrations analysed at the same time.

### DETAILED DESCRIPTION OF THE INVENTION

Unless defined otherwise, all technical and scientific terms used herein have the same meaning as commonly understood by a person skilled in the art related to this invention. Also, the singular forms “a”, “an”, and “the” are meant to include plural reference unless it is stated otherwise.

As mentioned above, the present invention relates to the determination of the active concentration of an analyte, typically a bioanalyte, in a fluid sample. In brief, the method is based on measuring analyte binding interaction at solid support surfaces or areas having different densities, or immobilization levels, of analyte-binding ligand, and determining from initial binding rate data the initial binding rate at complete transport limitation (i.e. where the interaction is diffusion-controlled), from which the active analyte concentration can be determined.

Preferably, an interaction analysis sensor is used in the active concentration determination, typically a biosensor.

Before describing the present invention in more detail, the concept of biosensors and the detection of interactions kinetics will be briefly described.

A biosensor is typically based on label-free techniques, detecting a change in a property of a sensor surface, such as mass, refractive index or thickness of the immobilized layer. Typical biosensors for the purposes of the present invention are based on mass detection at the sensor surface and include especially optical methods and piezoelectric or acoustic wave methods. Representative sensors based on optical detection methods include those that detect mass surface concentration, such as sensors based on reflection-optical methods, including e.g. evanescent wave-based sensors including surface plasmon resonance (SPR) sensors; frustrated total reflection (FTR) sensors, and waveguide sensors, including e.g. reflective interference spectroscopy (RIfS) sensors. Piezoelectric and acoustic wave sensors include surface acoustic wave (SAW) and quartz crystal microbalance (QCM) sensors.

Biosensor systems based on SPR and other detection techniques are commercially available. Exemplary such SPR-biosensors include the flow-through cell-based Biacore® systems (GE Healthcare, Uppsala, Sweden) and ProteOn™ XPR system (Bio-Rad Laboratories, Hercules, Calif., USA) which use surface plasmon resonance for detecting interactions between molecules in a sample and molecular structures immobilized on a sensing surface. As sample is passed over the sensor surface, the progress of binding directly reflects the rate at which the interaction occurs. Injection of sample is usually followed by a buffer flow during which the detector response reflects the rate of dissociation of the complex on the surface. A typical output from the system is a graph or curve describing the progress of the molecular interaction with time, including an association phase part and a dissociation phase part. This binding curve, which is usually displayed on a computer screen, is often referred to as a “sensorgram”.

With the Biacore® systems it is thus possible to determine in real time without the use of labeling, and often without purification of the substances involved, not only the presence and concentration of a particular molecule, or analyte, in a sample, but also additional interaction parameters, including kinetic rate constants for association (binding) and dissociation in the molecular interaction as well as the affinity for the surface interaction.

In the following, the present invention will to a large extent be described, for illustration only and no limitation, with reference to SPR sensors of the Biacore® system type.

As mentioned above, the Biacore® systems, as well as analogous sensor systems, measure the active analyte concentration as distinct from the total concentration of the analyte, the choice of ligand on the sensor surface defining the kind of activity being measured.

When an analyte is injected into a laminar flow system, e.g. of the Biacore® system type, in such a way that the analyte contacts a sensor surface, it will give rise to a binding event. The rate of the analyte/ligand interaction will be determined by the interaction kinetics and by the transport efficiency of the flow system.

For a biochemical interaction the rate at which an interaction proceeds is given by the difference between the forward (association) and the reverse (dissociation) processes. For a reversible 1:1 interaction between an analyte A and a surface-bound (immobilised) capturing molecule or ligand B which is not diffusion or mass transfer limited

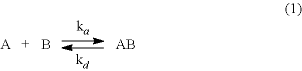

where kₐ and k_{d} are the rate constants for the association and dissociation, respectively.

The association rate is given by kₐ[A][B] and the dissociation rate is given by k_{d}[AB]. The net rate of binding (i.e. the change in surface concentration of formed complex B) is therefore

$$\frac{d [ AB ]}{d t} = {k}_{a} [ A ] [ B ] - {k}_{d} [ AB ] \qquad ( 2 )$$

After a time t, the concentration of unbound ligand B at the surface is [B_{T}]−[AB], where [B_{T}] is the total, or maximum, concentration of ligand B. Insertion into equation (2) above gives

$$\frac{d [ AB ]}{d t} = {k}_{a} [ A ] \{ [ {B}_{T} ] - [ AB ] \} - {k}_{d} [ AB ] \qquad ( 3 )$$

In terms of detector response units (in Biacore® systems formation of complex is observed as an increase in response, measured in resonance units (RU)), this can be expressed as

$$\frac{d R}{d t} = {k}_{a} C ( {R}_{max} - R ) - {k}_{d} R \qquad ( 4 )$$

were R is the response in response units, C is the concentration of analyte in the sample, and Rₘₐₓ is the response obtained if analyte (A) had bound to all ligand (B) on the surface, i.e. the maximum analyte binding capacity.

The kinetic rate constants kₐ and k_{d} are typically calculated by fitting response data for, preferably, a number of different concentrations of analyte and, preferably, also at least one other ligand density at the sensor surface to equation (4) above, or to the integrated form thereof:

$$R = \frac{{k}_{a} {CR}_{max}}{{k}_{a} C + {k}_{d}} ( 1 - {e}^{- ( {k}_{a} C + {k}_{d} ) t} \qquad ( 5 )$$

Software for the analysis of kinetic and affinity data is commercially available. Thus, for example, evaluation of kinetic and affinity data produced by the Biacore® instruments is usually performed with the dedicated BIAevaluation software (supplied by GE Healthcare, Uppsala, Sweden) using numerical integration to calculate the differential rate equations and non-linear regression to fit the kinetic and affinity parameters by finding values for the variables that give the closest fit, reducing the sum of squared residuals to a minimum. The “residuals” are the difference between the calculated and the experimental curve at each point, squared residuals being used to weight equally deviations above and below the experimental curve. The sum of squared residual is expressed by the equation:

$$S = {{\sum }_{l}}^{n} {( {r}_{f} - {r}_{x} )}^{2} \qquad ( 6 )$$

where S is the sum of squared residuals, r_{f} is the fitted value at a given point, and rₓ is the experimental value at the same point. For example, for the molecular interaction described above, such software-assisted data analysis is performed by, after subtracting background noises, making an attempt to fit the above-mentioned simple 1:1 binding model as expressed by equations (4) or (5) above to the measurement data.

Usually the binding model is fitted simultaneously to multiple binding curves obtained with different analyte concentrations C (and/or with different levels of surface derivatization Rₘₐₓ). This is referred to as “global fitting”, and based on the sensorgram data such global fitting establishes whether a single global kₐ or k_{d} will provide a good fit to all the data.

The above is, however, only valid for a reaction which is not diffusion or mass transfer limited.

Thus, for analyte to bind to the sensor surface, the molecule must be transported from the bulk solution to the sensor surface, which is a diffusion limited process. Under conditions of laminar flow, which apply in the Biacore® and analogous biosensor systems, the transport rate is proportional to the concentration of analyte in the bulk solution.

In a given analysis situation, the observed rate of binding at any time will be determined by the relative magnitudes of the net biochemical interaction rate and the rate of mass transport. If interaction is much faster than transport, the observed binding will be limited entirely by the transport processes. This is also the case when the analyte does not diffuse fast enough from the surface during dissociation, leading to re-binding. Conversely, if transport is fast and interaction is slow, the observed binding will represent the interaction kinetics alone. When the rates of the two processes are of similar orders of magnitude, the binding will be determined by a combination of the two rate characteristics.

The overall interaction process can be represented by the scheme

A_{bulk} [symbol] A_{surface}+B [symbol] AB (7)

The rate of mass transport from bulk solution to the surface is given by

$$\frac{d [ {A}_{surface} ]}{d t} = {k}_{m} [ {A}_{bulk} ] \qquad ( 8 )$$

where A_{surface} is the analyte concentration at the sensor surface, A_{bulk} is the analyte concentration in the bulk solution and kₘ is the mass transport coefficient.

The differential equation describing the binding interaction will therefore include a term for mass transfer of analyte to the surface corresponding to equation (8) above. For a flow cell, a “two-compartment” model consisting of a set of coupled ordinary differential equations and described in, for example, Myszka, D. G. et al. (1998) Biophys. J. 75, 583-594 is considered to give a reasonable description of the binding kinetics when the data are influenced by mass transport. In this model, the flow cell is assumed to be divided into two compartments, one in which the concentration of analyte is constant, and a second near the sensor surface where the analyte concentration depends on the mass transport rate, the surface density of ligand, and the reaction rate constants.

For the interaction exemplified above of a monovalent analyte A reacting with an immobilised monovalent ligand B, this model may be represented by the following two differential equations replacing equation (3) above

$$\begin{matrix}\frac{d [ {A}_{surface} ]}{d t} = ( - {k}_{a} {A}_{surface} ( {B}_{T} - AB ) + {k}_{d} AB + {k}_{m} ( {A}_{bulk} - A ) & ( 9 ) \\ \frac{d AB}{d t} = {k}_{a} {A}_{surface} ( {B}_{T} - AB ) - {k}_{d} AB & ( 10 )\end{matrix}$$

where kₘ is the mass transport coefficient (describing diffusive movement of analyte between the compartments), B_{T} is the total ligand concentration, A_{surface} is the concentration of free analyte at the sensor surface, A_{bulk} is the injection (i.e. initial) analyte concentration, AB is the concentration of complex AB (=surface density of bound analyte), and kₐ and k_{d} are the association and dissociation rate constants, respectively.

As will be described in more detail below, the mass transport coefficient kₘ can be calculated, and fitting of response data to equations (9) and (10) will give the kinetic rate constants kₐ and k_{d}.

The kinetic characterizations outlined above have traditionally been performed using either the well-established method where each sample concentration is run in a separate cycle, and analyte is removed by regeneration of the surface between each cycle. In a more recently developed approach, however, referred to as “single cycle analysis”, the analyte is injected with increasing (or otherwise varied) concentrations in a single cycle, the surface not being regenerated between injections. For a more detailed description of such single cycle analysis it may be referred to Karlsson, R., et al. (2006) Anal. Biochem. 349:136-147 (the disclosure of which is incorporated by reference herein).

For the above-mentioned mass transport coefficient kₘ, the following equation applies

$${k}_{m} = 0.98 \times {( \frac{D}{h} )}^{2 / 3} {( \frac{F}{0.3 \times w \times l} )}^{1 / 3} \qquad ( 11 )$$

where D is the diffusion coefficient (m²/s) of the analyte, F is the volumetric flow rate of liquid through the flow cell (m³/s), and h, w and l are the flow cell dimensions (height, width, length (m)).

The diffusion coefficient D is a function of the size and shape of the molecule and the frictional resistance offered by the viscosity of the solvent in question. For spherical molecules, the diffusion coefficient is inversely proportional to the radius and thus proportional to the cube root of the molecular weight. For very large solute molecules, such as proteins, however, the diffusion coefficient is relatively insensitive to the molecular weight.

If there is no literature value for the diffusion coefficient, it may determined experimentally, e.g. by analytical ultracentrifugation or light scattering. Alternatively, the diffusion coefficient may be estimated from the molecular weight and the shape factor, or frictional rate, according to the equation

$$D = 342.3 \times \frac{1}{{M}^{1 / 3} \times f \times {η}_{rel}} \times {10}^{- 11} \qquad ( 12 )$$

where D is the diffusion coefficient (m²/s), M is the molecular weight (daltons), f is the frictional ratio, and ηᵣₑₗ is the viscosity of the solvent relative to water at 20° C.

From equations (9) and (10), the mass transport coefficient kₘ can thus be calculated.

For Biacore® systems, a Biacore-specific mass transfer constant kₜ may be obtained by adjusting for the molecular weight of the analyte and conversion from measured response (in RU) to concentration units:

kₜ=kₘ×M×10⁹ (13)

where M is the molecular weight (Mw), and the conversion constant 10⁹ is approximate and only valid for protein analytes and a specific sensor surface, Sensor Chip CM5 (GE Healthcare, Uppsala, Sweden). For other analytes/sensor surfaces a calibration of the system with a RU conversion factor is required (1 RU equals×ng/mm²).

For evaluation of calibration-free concentration measurements, using the relationship between the diffusion properties of the analyte together with analysis of the binding rate under partially diffusion-controlled conditions, the mass transport coefficient is calculated from the diffusion coefficient, and then converted to the mass transport constant kₜ which is used in fitting the experimental data to the diffusion-controlled interaction model whereby the analyte concentration of the sample can be obtained.

Typically, the sample analyte, e.g. a protein, is run at two flow rates (e.g. 5 and 100 μl/min) against an immobilized ligand, the initial binding rate (dR/dt) of each run being determined (in the case of a Biacore® system, using SPR detection technology). The analyte concentration C is then evaluated (typically by dedicated software), setting the analyte concentration as a parameter to fit and kₘ as a known constant together with M_{W}.

Generally, binding rates should be measured shortly after start of the sample injection, since the rates approach zero as the binding approaches a steady state.

### The Invention

For a reaction that is totally limited by the transport efficiency of the system, the following equation applies

$$\frac{d R}{d t} = {k}_{m} C \qquad ( 14 )$$

where R is the detector response, kₘ is the mass transport coefficient, and C is the analyte concentration of the sample. That is, the response increase dR/dt, or binding rate, at the sensor surface is proportional to the mass transport coefficient and the active concentration of the analyte.

From equation (14) above it is seen that if the (initial) binding rate dR/dt at total transport limitation and kₘ are known, the analyte concentration C may be calculated by dividing dR/dt by kₘ.

For mass-sensing systems, however, like, for instance, the Biacore® systems, the response signals are mass-depending (RU for a Biacore® system), and the signal must be related to the mass according to equation (13) above. To obtain C, dR/dt is therefore divided by kₜ (rather than kₘ).

The present invention provides an approach to determining the initial binding rate at total transport limitation to thereby permit determination of active concentration. This is accomplished by measuring the initial binding rate at a number of different partially limited conditions obtained by using a plurality of different ligand densities on the sensor surface or surfaces.

More specifically, initial binding rates are measured at a number of, preferably at least four, different ligand density levels, i.e. immobilization levels. For each immobilization level, the response is recorded using at least one fixed flow rate. The initial binding rate is plotted versus the immobilization level, or maximal binding capacity Rₘₐₓ, and the binding rate where dR/dt has reached its maximum is determined by extrapolation of the data, typically using an algorithm capable thereof. This maximum binding rate corresponds to the binding rate at mass transport limitation, meaning that equation (14) above applies, and the active concentration C can therefore be calculated by dividing dR/dt by kt.

Extrapolation may, for example, be performed by fitting the data to the four-parameter regression equation conventionally used with Biacore™ systems:

$$Response = {R}_{high} - \frac{( {R}_{high} - {R}_{low} )}{1 + {( \frac{X}{{A}_{1}} )}^{{A}_{2}}} \qquad ( 15 )$$

where R_{high} and R_{low} are fitting parameters that correspond to the maximum and minimum response levels, respectively, A₁ and A₂ are additional fitting parameters, y is dR/dt and x is Rₘₐₓ or ligand density.

Other algorithms capable of identifying a maximum may also be applicable.

According to equation (11) above, the mass transport coefficient kₘ is proportional to (flow rate)^{1/3}. For a completely transport limited reaction, the binding rate dR/dt will therefore also be proportional to (flow rate)^{1/3} in accordance with equation (14).

In a, currently preferred, variant of the method, the analyte response is measured using two different flow rates. Data analysis can use the combined data for analysis as described below and, additionally, a plot of the binding ratio versus the immobilization level can be used to validate the mass transport criteria.

The plot of binding rate ratios can also be used to select data to be included in the concentration analysis as deviations from expected behaviour (overlapping graphs independent of dilution factor and predictable R_{high}) are hallmarks of this plot.

When binding data is collected over a wider range of immobilization levels, the fitting algorithm is then set up to identify the lowest immobilization level where the criterion of the binding rate being proportional to the cubic root of the flow rate is met. At this point, the active concentration can thus be derived as initial binding rate divided with the mass transport coefficient.

For example, assume that the flow rates are 10 μl/min and 80 μl/min, respectively. It is readily seen that for a reaction that is completely kinetically controlled, the binding rate will not be influenced by the flow rate, and the binding rate ratio will therefore be 1. In contrast, when the reaction is completely transport limited, the binding ratio will in this exemplary case be 2 [(80/10)^{1/3}]. The immobilization level (ligand density) for which the binding rate ratio equals 2 is thus determined and the analyte concentration may be calculated from either of the binding rates at that immobilization level.

In the latter method variant using two flow rates, the single cycle approach described above where the flow rate varies during one sample injection may be used. For instance, the sample may be injected at 10 μl/min and after 5-20 seconds the flow rate is increased to 100 μl/min. In data analysis, this is handled by input of actual flow rates in the evaluation algorithms used.

Optionally, the method is performed with two or more different dilutions of a liquid sample.

As mentioned above in connection with Equation (13), the RU conversion factor is given for Sensor Chip CM5. If the concentration has been determined on a CM5 surface, the same concentration should be obtained with a different surface, but with a different conversion factor depending on e.g. the distance from the measuring surface. By performing a global analysis on a CM5 surface, the mass transport constant kₜ for other surfaces may be calculated and the field of use thus be extended to other types of surfaces.

The contacting of the sample with surfaces or surface areas with different ligand densities may be performed in an analytical instrument having a single sensor surface by sequential sample injections and varying the ligand density between injections. Preferably, however, the method is performed with a multi-flow channel instrument, such as the Biacore® T100, T200 or 4000 (GE Healthcare, Uppsala, Sweden) or ProteOn™ XPR system (Bio-Rad Laboratories, Hercules, Calif., USA), preferably by parallel sample injections.

Optionally, when binding data is collected on different immobilization levels and at different flow rates, the method of the invention may be combined with “conventional” calibration-free concentration analysis (CFCA) as described above. Such combined analysis thus will take into account both extrapolation to complete transport limitation (as in the method of the invention) and analysis under partial transport limitation (as in “conventional” CFCA) and therefore has the potential to become more robust.

The combined analysis is possible by globally fitting common parameters. In conventional CFCA C is fitted directly. In the 4-parameter fit based on equation (15) above, R_{high} is determined and the corresponding binding rate dR/dt is divided by kₜ to obtain the analyte concentration C as described above. Here, the concentration C may alternatively be set as a fitted parameter in a modified 4-parameter-equation. This means that kinetic based and initial binding rate experiments share a global parameter so that experiments can be co-evaluated.

In the combined analysis, the “conventional” CFCA is preferably replaced by an improvement of the latter where several different dilutions of a liquid sample are used, and at least some of dilutions are included in a global fit of concentration data, the same fitting criteria being applied to several dilutions. Thereby, the analysis is made more robust and the dynamic range of the analysis will be extended. Such improved CFCA is described in our co-pending application “Method of determining active concentration by calibration-free analysis” filed on even date with the present application (the disclosure of which is incorporated by reference herein).

In an advantageous method of determining active concentration in a calibration-free format, (initial) binding rate data are collected at different immobilization levels and at different flow rates at partial or complete transport limitation for a number of different sample dilutions. One then has the choice to evaluate the obtained binding rate data according to several alternatives, i.e. either (i) by extrapolation to complete transport limitation; or (ii) by analysis under partial transport limitation, preferably using the improved CFCA with global fitting of data for several dilutions of a sample; or (iii) a combination of (i) and (ii).

When binding data are generated for a plurality of ligand densities (immobilization levels) and different flow rates, and a constant mass transport constant kₜ is used for the fittings, it is possible to fit concentration and kinetics simultaneously with data from one and the same experiment.

Concentration analysis and kinetic analysis may be combined in various ways. For instance, in a capture experiment, antibodies may first be captured on the surface and their concentration be determined by CFCA, after which the experiment will continue with “normal” kinetic analysis through injection of the antigen. A single experiment will then give both antibody concentration and antibody kinetics.

The invention will now be illustrated further by means of the following non-limiting examples.

### EXAMPLES

### Example 1

A procedure for determining active concentration by the method of the present invention using, for example, a modified Biacore® T200 system may be performed as follows:

(Note that this description is focused on the CFCA experiment itself and that other assay development steps, such as selection of immobilization method, buffer conditions and regeneration conditions, have already been performed.)

1) Immobilize the ligand on discrete spots/channels on the sensor surface using a suitable immobilization technique (amine coupling, thiol coupling, biotinylation and capture on streptavidin or by immobilizing other tag or domain specific capture reagents). Note the immobilization levels.
2) Leave one spot without immobilized ligand for referencing.
3) Prepare serial dilutions (typically in steps of two to five) of the sample.
4) Use the Method Builder software to set up the assay with the following assay steps:
a) startup cycles used for conditioning of the system Startuo cycles are typically mimics of the sample cycles but are disregarded in data analysis;
b) sample cycles for the actual analysis.

In each cycle provide the software with relevant inputs to define conditions for the injection of the sample (flow rate, spots to reach during injection, contact time, dissociation time and inject quality). In case a reversible capture immobilization is used an additional injection is used to capture the ligand of interest (for instance a histidine tagged protein can be captured on an immobilized anti histidine antibody). In most cases a regeneration solution is also programmed.

Typical program inputs for the sample are:

High performance inject, Flow path 1, 2, Contact time 36 s, Dissociation time 5 s, Flow rate 5 or 100 μl/min. The sample name and a dilution factor are also provided. Preferentially, at least one injection of the sample at zero concentration should be programmed.

(Note that variation of flow rate during injection is not possible using the commercial software).

5) Choose a high data collection rate—typically 10 Hz.
6) Run the assay.
7) Repeat the assay using at least four different ligand densities.
8) Open the data in a suitable evaluation program, for instance, Biacore® T200 evaluation software or Biaevaluation software 4.1.
9) Select an appropriate blank injection (sample at zero concentration).
10) Subtract the blank injection from the sample injection.
11) Display the data in an overlay plot.
12) Identify the earliest part of the binding curve that is unaffected by remaining referencing or blank subtraction artefacts. At a flow rate of 5 μl/min this is usually a few seconds into the injection, and at 100 μl/min typically close to one second into the injection.

Further analysis is divided into parts.

### Part 1

### Determination of Concentration Using Initial Binding Rates

1) Select a window of the earliest undisturbed data to determine the initial binding rate. A typical data window is five to ten seconds but can be shorter or longer.
2) Fit the data to a linear equation y=a*x+b, where x corresponds to the initial binding rate.
3) In the case where the flow rate is varied during the injection, select data for the second flow as early as possible after the change of the flow rate and determine the initial binding rate for the second flow rate.
4) Calculate the ratio of binding rates obtained at different flow rates.
5) Prepare graphs—for each sample dilution—where the initial binding rate and the ratio of binding rates described in point 4 are plotted versus the level of immobilized ligand.
6) Fit the binding rate data using equation (15) above either directly which will return a maximum binding rate for each sample dilution (all fitted parameters are local) or preferably to a modified version where R_{high} is substituted with parameters that account for sample dilution and flow rate variations. For instance, instead of R_{high}, R_{high}*((Flrate/Lfrate)̂(⅓))/Dil can be used, where Flrate is the actual flow rate at which the binding rate is determined, Lfrate is the lowest flow rate used in the experiment, and Dil is the dilution factor for the sample.
7) Obtain the concentration of the sample by dividing R_{high} with the kₘ value.

Note that when the global fit is used, the kₜ value to divide with is the one corresponding to the lowest flow rate.

### Part 2

### Single Cycle CFCA

In this approach the flow rate is changed during the injection. By linking the time segments to their respective flow rates this data can readily be analysed.

### Part 3

### Global Analysis of Conventional CFCA Data.

This procedure incorporates a modification of the CFCA algorithms to allow global analysis of several concentrations. This is done by deriving local concentrations through a dilution factor and by performing a global fit.

### Example 2

### Simulations of Initial Binding Rates

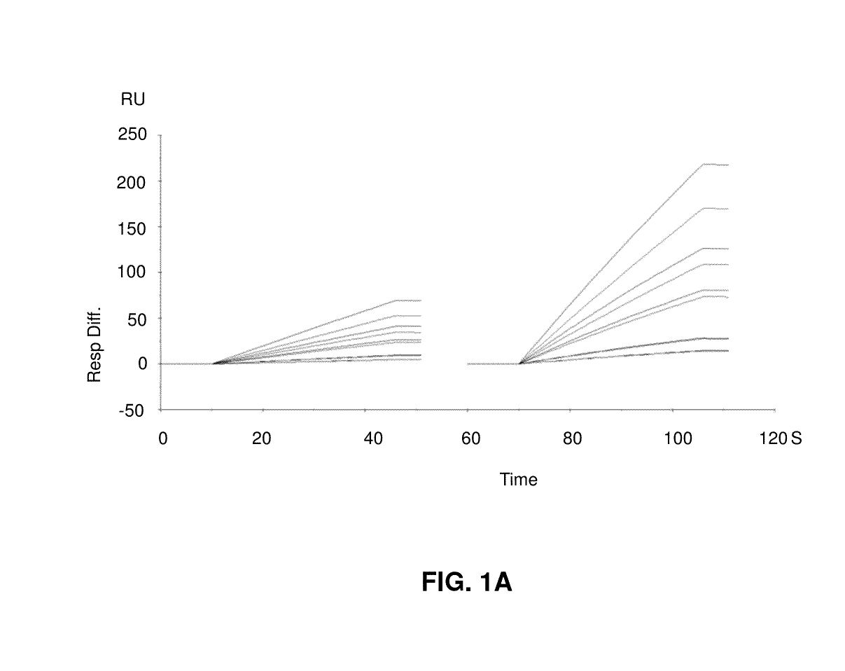

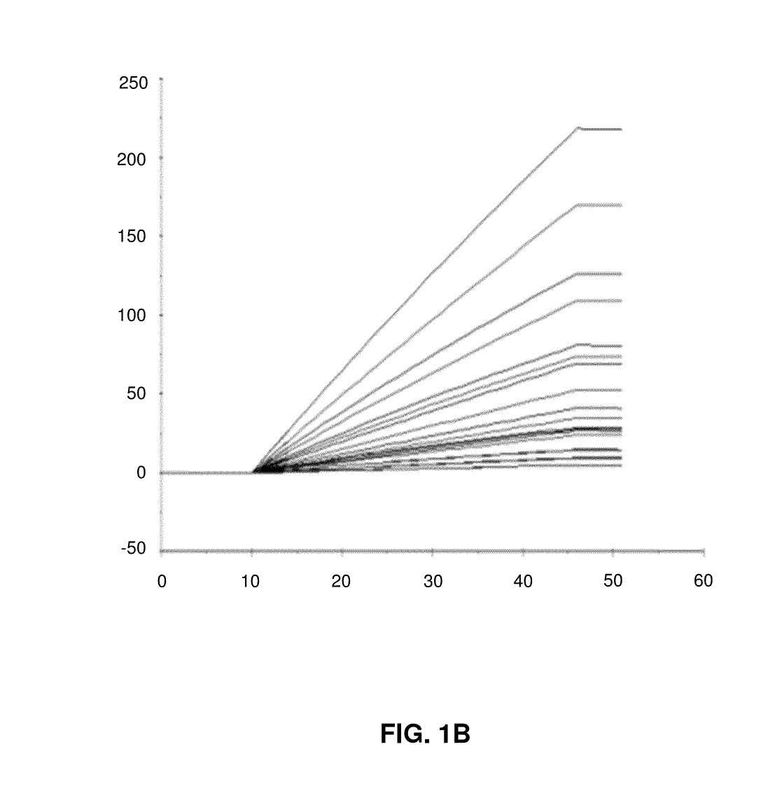

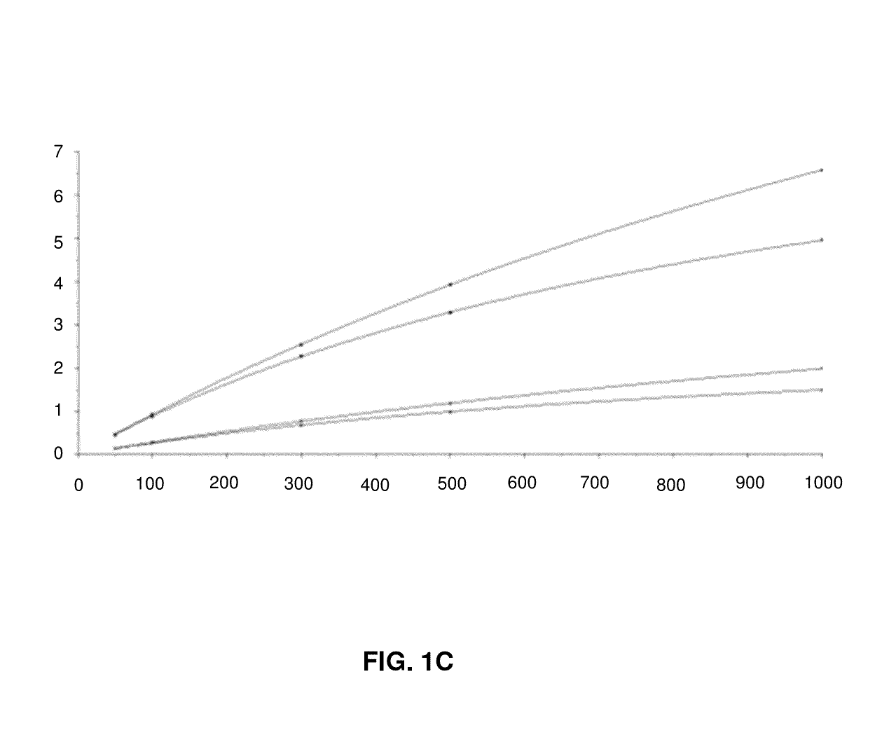

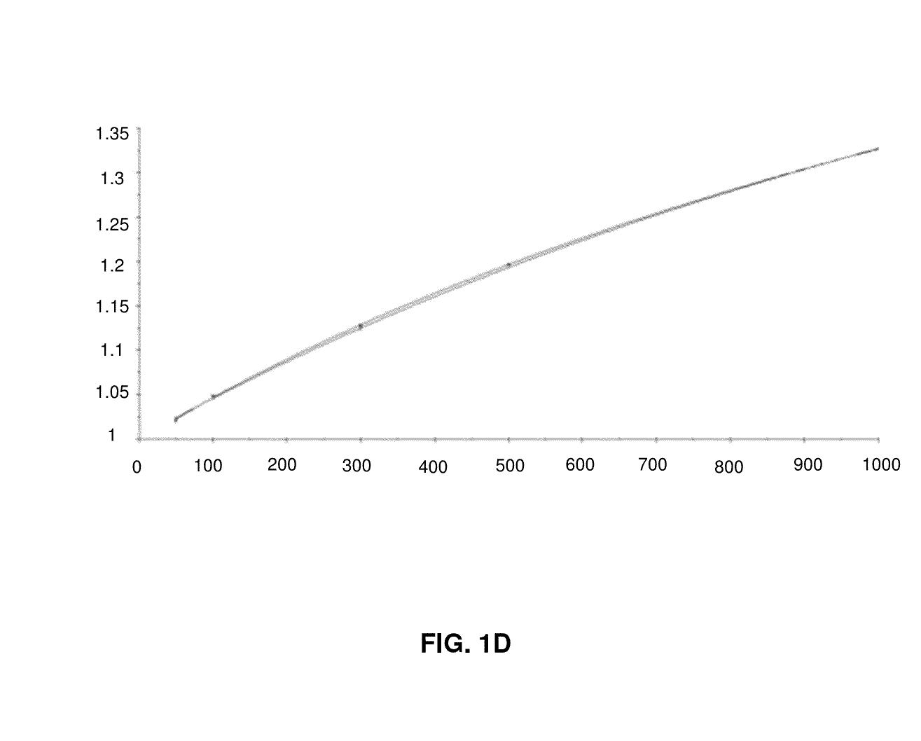

In the following, example data from simulations of initial binding rates will be described with reference to FIGS. 1A to 1D.

FIG. 1A shows overlay plots of simulated data. In the left panel, the concentration is 3 nM (dilution factor 10). Ligand levels giving rise to binding capacities of 50, 100, 300, 500 and 1000 RU, ka 1e6, kd 1e-3, kt 1e9 (red) or 2e9 (blue). In the right panel, the concentration is 10 nM (dilution factor 3). All other parameters are as in the left panel.

FIG. 1B shows a linear fit of binding data with a 3 seconds window 1 to 4 seconds into the injection.

FIG. 1C shows a global fit using a modified 4-parameter equation. Binding rates for each concentration at both flow rates are plotted versus ligand density (Rmax). Data returned by analysis: Rhi, Chî2, concentration of undiluted sample 302,62E-06 30 nM.

FIG. 1D shows binding rate ratio plotted versus ligand density (Rmax). Note that data overlap. Rhi fitted is 2.08 consistent with a two fold change in kt.

### Example 3

### Analysis of Binding Data Obtained with Immobilized Anti Beta-2-Microglobulin Antibody and Beta-2-Microglobulin

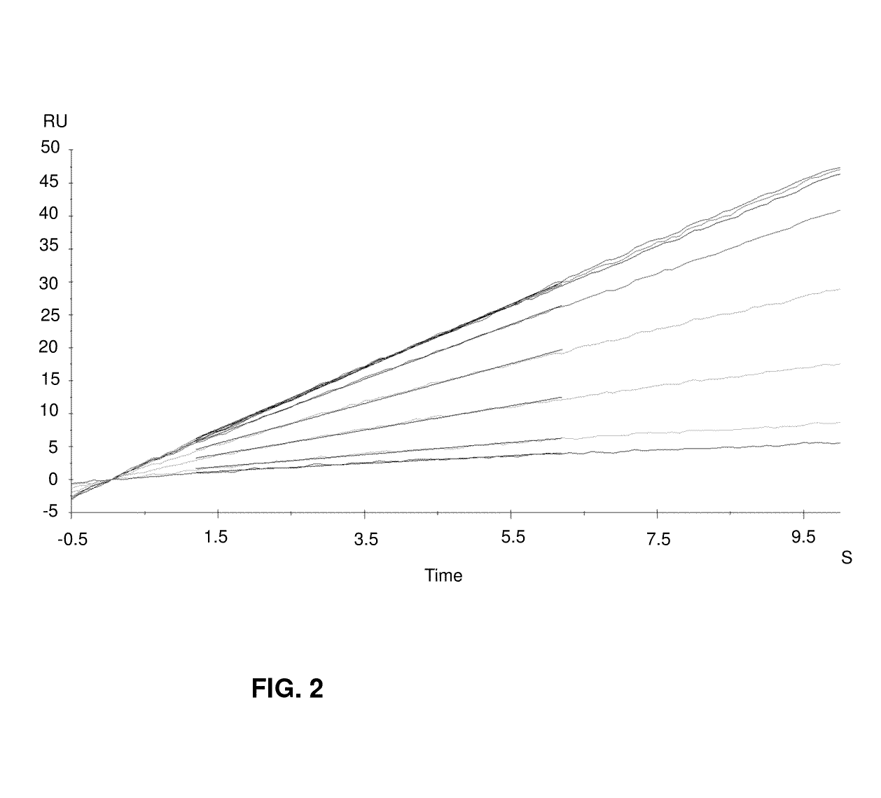

In this example the antibody was immobilized at eight different binding levels (ranging from 435 to 13300 RU), and one concentration of beta-2-micro-globulin was injected at a flow rate of 5 μl/min. The binding data and a linear fit to data over a 5 s window are demonstrated in FIG. 2.

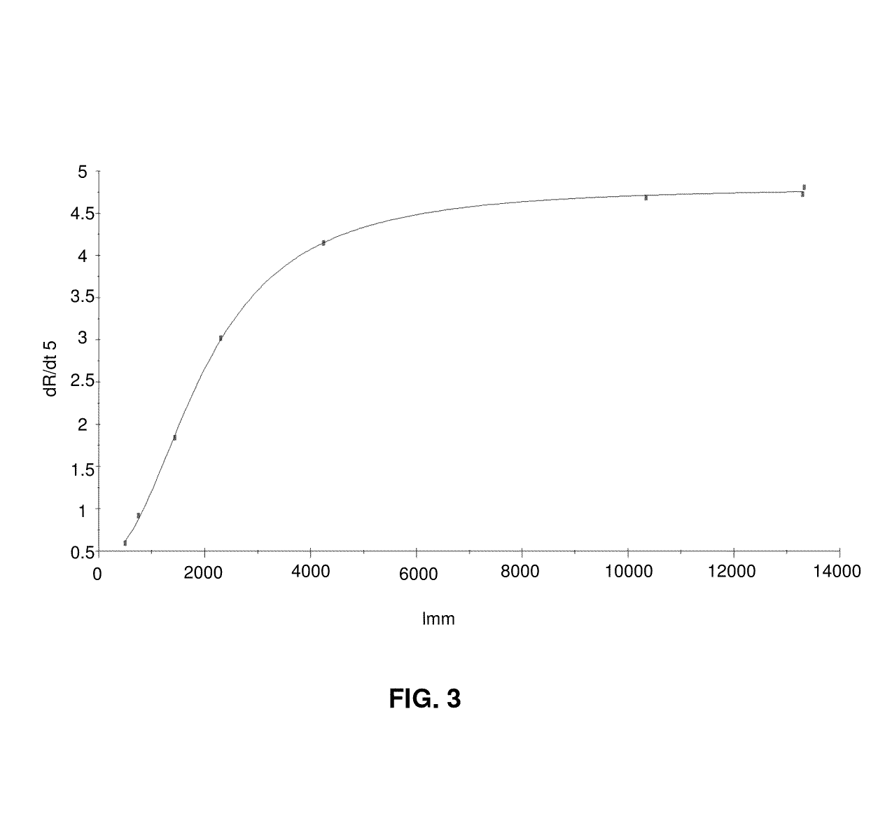

The initial binding rates (dR/dt) were plotted versus immobilization level (1 mm) and a fit of the data using equation (15) is illustrated in FIG. 3.

The concentration of beta 2 microglobulin was determined by dividing Rhi (in this case 4.81 RU/s with the relevant kt value 4, 9e8 RU/(M*s) giving an active concentration of 9.9 nM.

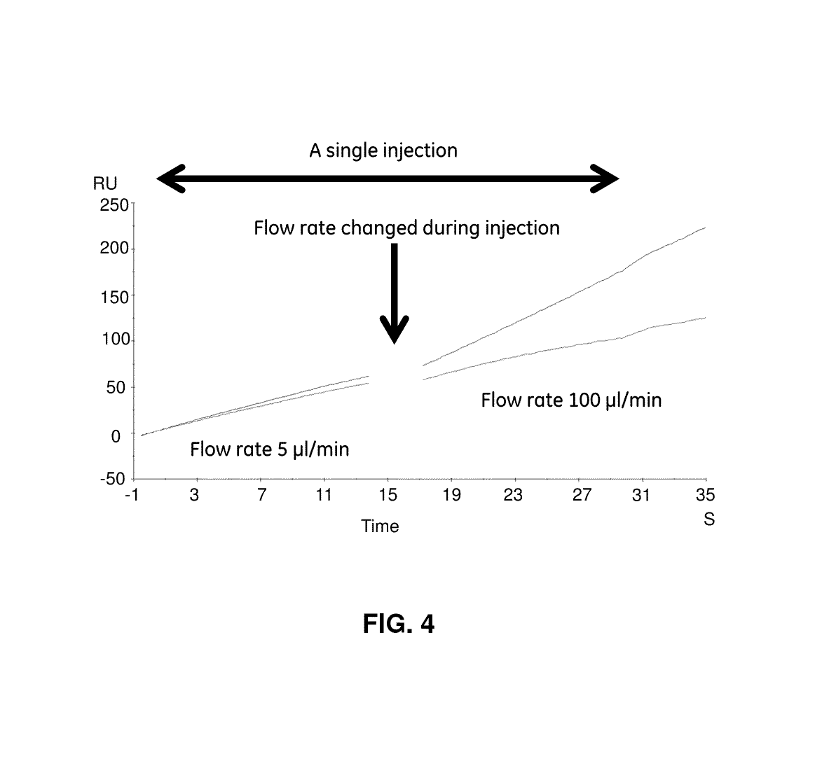

Data obtained at immobilization levels 4200 and 10400 RU were analysed at two flow rates as illustrated in FIG. 4. In this experiment the flow rate was initially five μl/min, and 15 seconds into the injection it is changed to 100 μl/min. An offset in the signal of a few RU has been cut away. The increase in flow rate is accompanied by an increase in the binding rate.

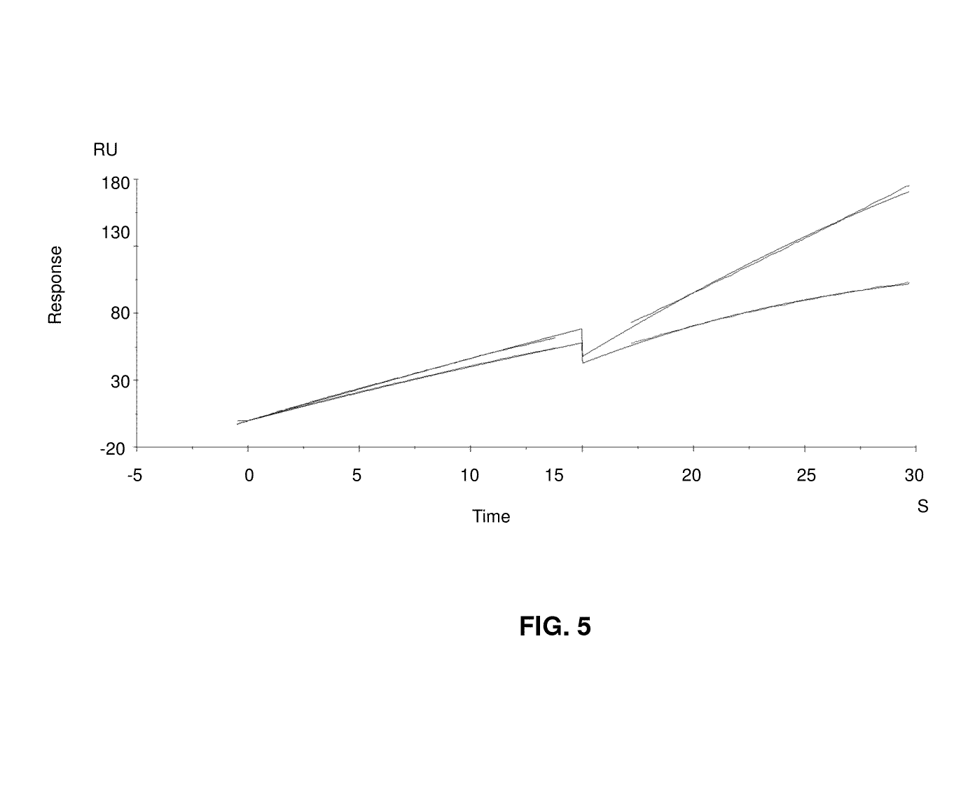

This data as shown was analysed directly without reference and blank subtraction, as shown in FIG. 5.

The concentration determined by this procedure was 10.7 nM and additional parameters used in the fit and obtained during the fit are given below in Table 1 below.

TABLE 1

| | Conc | SE(Conc) | Rmax | ton1 | tonoff | toff2 | kt1 | kt2 | offset |
|---|---|---|---|---|---|---|---|---|---|
| | 1.07E−08 | 5.94E−11 | | | | | | | |
| Immob level 10338 RU | | | 3.96E+02 | 0 | 15 | 30 | 4.89E+08 | 1.33E+09 | −20.9 |
| Immob level 4241 RU | | | 179 | 0 | 15 | 30 | 4.89E+08 | 1.33E+09 | −15.3 |

Fitted Rmax values correspond to immobilization levels while other parameters were used as constants. kt1 and kt2 are the transport coefficients at 5 and 100 μl/min, respectively.

### Example 4

### Global Fit of CFCA Data

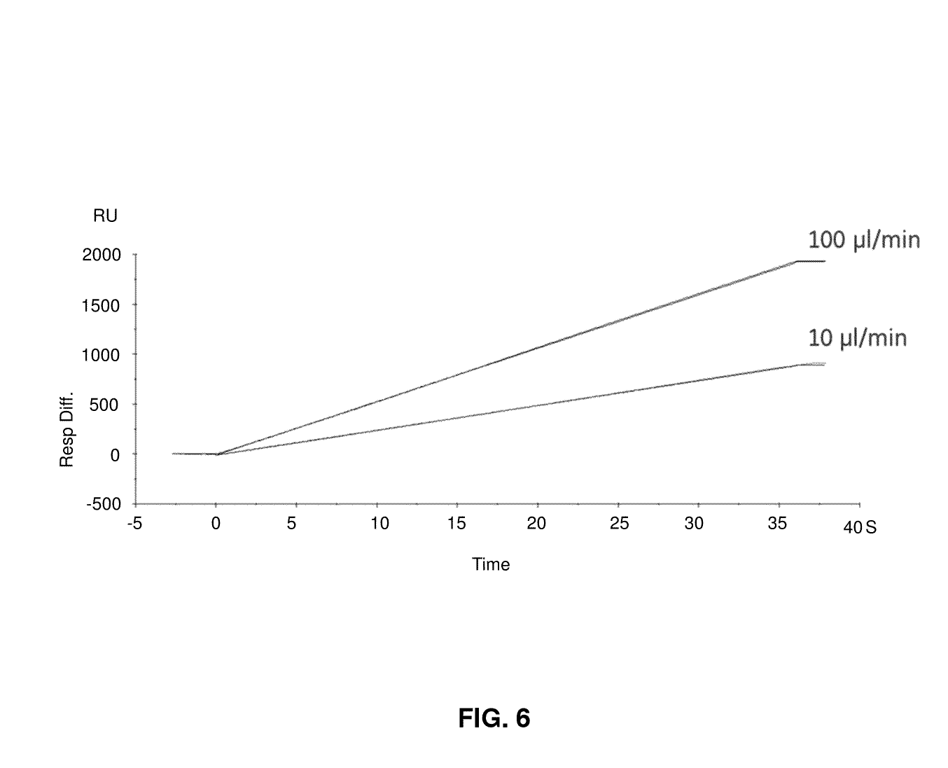

The experiments described here demonstrate antibody binding to an immobilized protein A derivative capable of binding antibody. The conventional CFCA analysis is illustrated in FIG. 6.

The analyte was injected in separate cycles at varying flow rates. In this case the analyte was diluted 400 times relative to its stock concentration. CFCA analysis gives the local antibody concentration as 19.1 nM and thus the stock solution is 7.6 μM.

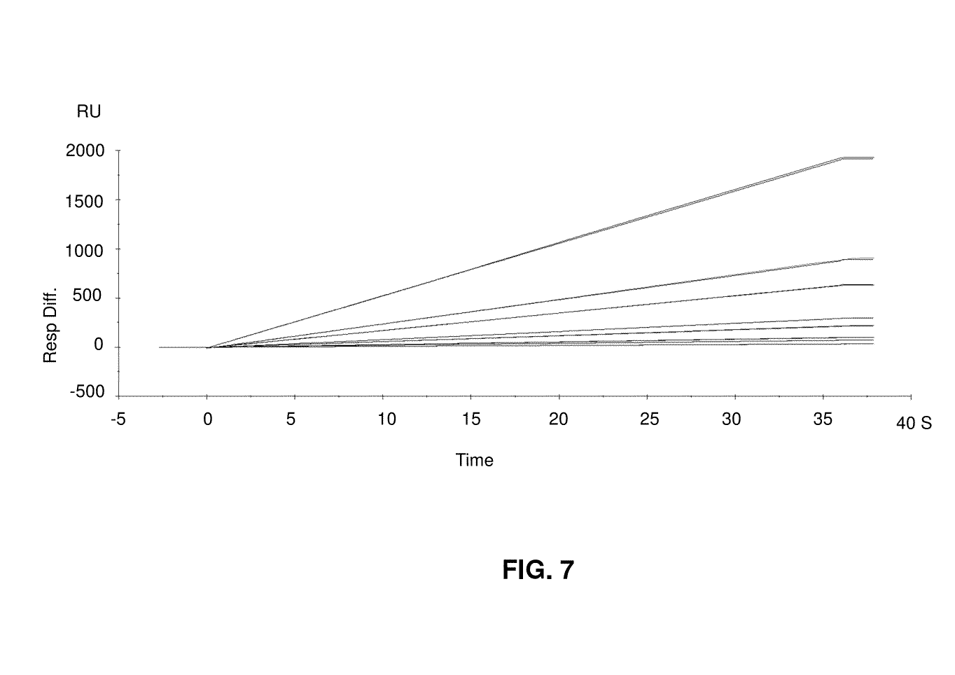

In many cases, however, it is useful to test several dilutions of the sample and analysis becomes tedious. By turning to a global fit of the data several concentrations are analysed at the same time, as shown in FIG. 7.

This graph illustrates the global fit of antibody dilutions 1:400, 1:1200, 1:3600 and 1:10800. Each dilution is injected at two flow rates and the globally determined concentration for the stock solution is 7.6 μM. This immediately demonstrates that the concentration analysis is not dilution dependent and simplifies the analysis.

The present invention is not limited to the above-described preferred embodiments. Various alternatives, modifications and equivalents may be used. Therefore, the above embodiments should not be taken as limiting the scope of the invention, which is defined by the appending claims.

1. A method of determining active concentration of an analyte in a sample, comprising the steps of:
(a) contacting a laminar flow of the sample with a plurality of solid phase surfaces or surface area supporting a ligand capable of specifically binding the analyte, each surface or surface area having a different ligand density;
(b) determining the initial binding rate (dR/dt) of analyte to the ligand at each ligand-supporting surface or surface area;
(c) determining from the determined initial binding rates the initial binding rate corresponding to transport-limited interaction at the surfaces or surface areas; and
(d) determining from the initial binding rate determined in step (c) the active analyte concentration.

2. The method of claim 1, wherein the binding rates are determined at least four different ligand densities.

3. The method of claim 1, wherein the initial binding rate corresponding to transport-limited interaction is the maximum initial binding rate.

4. The method of claim 1, wherein at least two different flow rates are used.

5. The method of claim 4, which comprises determining the initial binding rates for two different flow rates, from initial binding rate ratios at the different ligand densities determining the lowest ligand density where the initial binding rate is proportional to the cubic root of the flow rate, and from this initial binding rate determining the active analyte concentration.

6. The method of claim 4, further comprising using a plot of the binding ratio at the different flow rates versus the immobilization level to validate mass transport criteria.

7. The method of claim 4, further comprising using a plot of the binding ratio at the different flow rates versus the immobilization level to select data to be included in concentration analysis.

8. The method of claim 4, wherein the flow rate is varied during a single contacting cycle.

9. The method of claim 1, further comprising co-evaluating data from determining active concentration by kinetic analysis at different flow rates under partial or complete transport limitation, where binding data are fitted to a kinetic model where the transport coefficient is constant.

10. The method of claim 9, wherein several dilutions of the liquid sample are used and included in a global fit of the binding data.

11. The method of claim 1, wherein an interaction analysis sensor is used, preferably a biosensor.

12. The method of claim 11, wherein the interaction analysis sensor is based on mass-sensing, preferably evanescent wave sensing, especially surface plasmon resonance (SPR).

13. The method of claim 1, which is computer-implemented.

14. A computer program product comprising instructions for causing a computer to perform the method steps of claim 1.
